(19) United States
(12) Patent Application Publication
(10) Pub. No.: US 2007/0249677 A1
(43) Pub. Date: Oct. 25, 2007

(54) Treatment of Osteoarthritis and Dosing Regimen for Arzoxifene

(76) Inventors: Charles Thomas Benson, Hampshire (GB); Kristine Denisse Harper, Indianapolis, IN (US); Bruce Howard Mitlak, Indianapolis, IN (US); Yanfei Linda Ma, Carmel, IN (US)

Correspondence Address: ELI LILLY & COMPANY, PATENT DIVISION, P.O. BOX 6288, INDIANAPOLIS, IN, 46206-6288, US

(21) Appl. No.: 11/575,429

(22) PCT Filed: Oct. 13, 2005
(86) PCT No.: PCT/US05/36662

§ 371 Date: Mar. 16, 2007

Related U.S. Application Data
(60) Provisional application No. 60/620,202, filed on Oct. 19, 2004.

(51) Int. Cl. A61K 31/453

(52) U.S. Cl. 514/320

(57) ABSTRACT

The present invention provides a method for treating osteoarthritis in a mammal, comprising administering to a mammal in need thereof, an effective amount of a compound of Formula (I) or a pharmaceutically acceptable salt or solvate thereof.

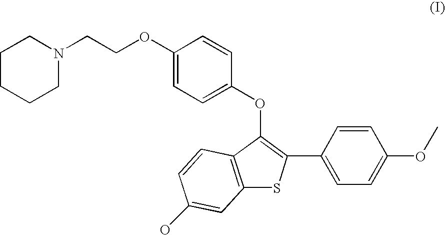

### FIELD OF THE INVENTION

The present invention relates to the use of 2-(4-Methoxyphenyl)-4-[4-[2-(1-piperidinyl)ethoxy]phenyoxy]benzo[b]thiophene-6-ol for the treatment of osteoarthritis. The compound 2-(4-Methoxyphenyl)-4-[4-[2-(1-piperidinyl)ethoxy]phenyoxy]benzo[b]thiophene-6-ol is also known as arzoxifene (hereinafter “arzoxifene”).

The present invention further provides a once-weekly and twice-weekly dosing regimen for arzoxifene.

### BACKGROUND OF THE INVENTION

The compound, arzoxifene, employed in the method of the present invention is known. The compound, methods of preparing the compound, as well as pharmaceutical formulations containing the compound, are described in U.S. Pat. No. 5,723,474 (herein “'474 patent”). The '474 patent discloses that arzoxifene can be useful for the treatment of the various medical indications associated with post-menopausal syndrome, and uterine fibroid disease, endometriosis, and aortal smooth muscle cell proliferation. “Post-menopausal syndrome” is a term used to describe various pathological conditions frequently affecting women who have entered into or completed the physiological metamorphosis known as menopause. Although numerous pathologies are contemplated by the use of this term, three major effects of post-menopausal syndrome are the source of the greatest long-term medical concern: osteoporosis, cardiovascular effects such as hyperlipidemia, and estrogen-dependent cancer, particularly breast and uterine cancer. Post-menopausal osteoporosis is a net loss of bone mass per unit volume resulting from a lack of endogenous estrogen occurring in a woman following the cessation of menstruation due to natural or surgical processes.

Applicants are aware of no reports of the use of arzoxifene to modulate the experimental models of osteoarthritis. More particularly, there are no reports describing the use of arzoxifene for treatment of osteoarthritis in clinical patients.

Osteoarthritis (hereinafter “OA”) is a chronic degenerative disease affecting the joints. Due to the involvement of the hip and knee, OA causes substantial pain, functional limitation, disability, and requirement for medical treatment, including joint replacement surgery. Although OA is more common among women, OA was not shown to be associated with the age of menarche or menopause or use of oral contraceptives in a study of British women (Samanta A, et al. “Is osteoarthritis in women affected by hormonal changes or smoking”, Br. J. Rheumatol 1993; 32:366-70. OA reportedly affected 20.7 million people, or 12.1% of U.S. adults in 1990. The number of people affected by OA is now estimated to be 37 million, trailing chronic heart disease as the leading cause of Social Security payments due to long-term absence from work. Lawrence R C, et al. Arthritis and Rheumatism 1998; 41:778-799.

There is no definitive answer regarding the cause of OA. OA is thought to be the result of decreased production and increased degradation of the cartilaginous matrix; however, the definitive cause of the degradation is not known. Further, there is no marketed drug that claims to reverse osteoarthritis. Most therapeutic agents are directed at reducing the inflammation and relieving the pain associated with OA. Non-steroidal anti-inflammatory drugs (NSAIDs) are typically the first line of treatment for OA, but long-term use may lead to gastric ulcers, kidney damage, and may even inhibit cartilage formation. Other treatments currently used to alleviate the debilitating effects of OA include surgical joint replacement, such as hip and/or knee replacement.

Thus, there is a need for development of new pharmaceutical compounds that can be used to treat OA. Compounds offering a desirable side effect profile, while slowing or reversing OA-related signs and symptoms would be especially desired. Arzoxifene may address the need for a non-surgical treatment offering an acceptable safety profile and clinical relief for the patient suffering from OA. Further, arzoxifene may be able address the need for a treatment that may slow or reverse the progression of OA.

The '474 patent teaches that arzoxifene can be administered using a daily dosing regimen. The '474 patent states that cyclical therapy using arzoxifene may be especially useful for the treatment of endometriosis or acutely during painful attacks of the disease. Further, the '474 patent states that in the case of restenosis, therapy may be limited to short intervals (1-6 months) following medical procedures such as angioplasty. However, daily dosages and daily dosing regimens are stated for the treatment of osteoporosis, on-going treatments, and other chronic conditions. Animal studies using arzoxifene support that the half life of arzoxifene is consistent with a daily dosing regimen.

Many patients prefer to take medications only once-weekly for increased convenience, improved dosing compliance, and to maximize patient safety. The present invention fulfils the patient's desire for a convenient once-weekly or twice-weekly arzoxifene dosing regimen providing a pharmaceutically acceptable safety profile.

### BRIEF DESCRIPTION OF THE FIGURES

FIG. 1 illustrates the general location of the Zones in the knee joint used to measure OA progression.

FIG. 2 intentionally omitted.

FIG. 3 is an image analysis of OA data.

FIG. 4 is a histological analysis of OA data.

FIG. 5 is a histological analysis of OA data.

FIG. 6 is a histological analysis of OA data.

FIG. 7 is a histological analysis of OA data.

FIG. 8 is a histological analysis of OA data.

FIG. 9 is a summary of a less than daily dosing data.

As indicated in the above graphs, legends, and figures, the term “353381” and “LY353381” is Arzoxifene.

### SUMMARY OF THE INVENTION

The present invention provides a method for treating osteoarthritis in a mammal, comprising administering to a mammal in need thereof, an effective amount of a compound of Formula I: 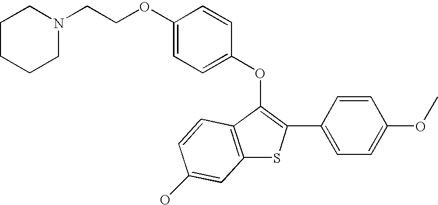
or a pharmaceutically acceptable salt or solvate thereof.

In another embodiment of the present invention, a method is provided for treating osteoarthritis comprising administering to a mammal (including a human) in need of such treatment, an effective amount of arzoxifene.

The present invention also provides the use of arzoxifene for the manufacture of a medicament for treating osteoarthritis in a patient in need thereof.

Furthermore, the present invention provides the use of arzoxifene for treating osteoarthritis in a patient in need thereof.

In another embodiment of the present invention, a once weekly dosing regimen is provided. In a further embodiment of the present invention, a twice-weekly dosing regimen is provided.

A further embodiment is the use of arzoxifene for breast cancer risk reduction, comprising administering a less than daily dosing regimen of an effective amount of arzoxifene to a patient in need thereof.

A further embodiment is the use of arzoxifene for the treatment of osteoporosis comprising administering a less than daily dosing regimen of an effective amount of arzoxifene to a patient in need thereof.

A further embodiment is the use of arzoxifene for the preservation of bone mineral density comprising administering a less than daily dosing regimen of an effective amount of arzoxifene to a patient in need thereof.

### DETAILED DESCRIPTION

The present invention relates to a method for treating osteoarthritis in a mammal in need thereof. The method of the present invention comprises administering to a mammal in need thereof, a pharmaceutically effective amount of arzoxifene. Further, the present invention comprises administering a pharmaceutically effective amount of arzoxifene as a unit dosage, wherein said dosage is administered according to a continuous schedule having a dosing interval selected from the group consisting of once-weekly dosing and twice-weekly dosing. In other embodiments, the present invention relates to methods comprising a continuous dosing schedule having a dosing periodicity ranging from about once every 2 days to about once every 5 days.

The present invention utilizes higher unit dosages of aroxifene at each dosing point than has heretofore been typically administered; however, due to the dosing schedule provided herein, patient safety is preserved and may be further enhanced by less frequent dosing. Moreover, the method is more convenient for the patient.

The methods of the present invention are administered to mammals in need of such treatment. Generally, it is preferred that the mammals are human patients. However, for veterinary science use, the methods of this invention can be useful for mammals in need of OA treatment wherein the mammals are horses or companion animals. It is generally preferred that the mammals are human patients in need of OA treatment. In another embodiment, it may be preferred that the OA condition is diagnosed by a qualified health professional.

As used herein, the term “pharmaceutically effective amount” or “effective amount” means that amount of arzoxifene, or a salt or solvate thereof, that will elicit the desired therapeutic effect when administered using a dosing regimen. An effective amount for the treatment of OA is an amount that slows or reverses the signs and symptoms of OA. An effective amount of arzoxifene is an amount capable of inhibiting or preventing the structural progression and/or symptoms of OA in a patient in need thereof. Further, as used herein, “effective amount” refers to an amount of arzoxifene, or a salt or solvate thereof, capable of treating osteoporosis, preserving bone mineral density, or reducing the risk of breast cancer, respectively, in a patient in need thereof.

The term “continuous” dosing or continuous schedule, as used herein, means that the dosing regimen is repeated until the desired therapeutic effect is achieved. The continuous schedule or continuous dosing schedule is distinguished from cyclical or intermittent administration.

The term “less than daily dosing” means any intentional dosing regimen comprising at least one period in which the frequency of dosing is less than daily. For example, less than daily dosing may be every other day, or comprising at least one gap of more than one day where there is no administration of arzoxifene. In the case of dosing regimens that comprise a gap or day without administration, as used herein, such gaps or days without arzoxifene administration must be preceded and followed by administration of arzoxifene, once the initial dose has been administered. The term less than daily dosing is meant to include any dosing regimens comprising for example, five days with and two days without administration of arzoxifene, three days with and three days without, three days without and one day with, two days without and one day with, four days without and one day with, and the like. This is true regardless of the average number of doses per day. For example, twice-daily administration every other day or every third day would each be less than daily dosing. As used herein, a day with dosing or a day with administration of arzoxifene includes once, twice, or any number of doses on a particular day. As used herein the term “less than daily dosing” shall mean that arzoxifene is administered at least once each week or one time during each sequential ten (10) day period. That is, at least twice monthly dosing of arzoxifene is contemplated by the term “less than daily dosing”.

The term “solvate” refers to an aggregate that comprises one or more molecules of the solute, such as an aggregate of compound I, with one or more molecules of solvent. Suitable solvent molecules are those commonly used in the pharmaceutical art, which are known to be non-detrimental to the recipient, e.g., water and ethanol. The preparation of a solvated form of arzoxifene has been described in the art.

Although the free-base form of arzoxifene can be used in the formulations and methods of the present invention, preferably, the compounds are in the form of a pharmaceutical salt. Thus, the term “pharmaceutically acceptable salt” refers to acid addition salts of compound I which are substantially non-toxic at the doses administered and are commonly known in the pharmaceutical literature. See e.g. Berge, S. M, et al., J. Pharm. Sci., 66(1), (1977). Pharmaceutically acceptable salts of arzoxifene are described in the '474 patent.

As used herein, the term “inhibiting bone resorption” means treating or preventing bone resorption by the direct or indirect alteration of osteoclast formation or activity. Inhibition of bone resorption refers to treatment or prevention of bone loss, especially the inhibition of the removal of existing bone either from the mineral phase and/or the organic matrix phase. The term “preserving bone mineral density”, as used herein, means that the bone mineral density is increased, maintained, and/or reduced rate of loss of bone mineral density, in a patient in need thereof. Bone mineral density can be measured using methods well known in the art. For example, bone mineral density can be assessed using posterior anterior lumbar spine and femoral neck bone and/or hip mineral density measurements using DXA screening.

As used herein, the term “abnormal bone resorption” means a degree of bone resorption that exceeds the degree of bone formation, either locally, or in the skeleton as a whole.

The dosage to be administered may vary depending upon the physical characteristics of the patient, the severity of the patient's symptoms, and the means used to administer the drug. The specific dose for a given patient is usually set by the judgment of the attending physician.

Arzoxifene can be administered on a daily basis for the treatment of OA. A typical daily dose of arzoxifene would contain a nontoxic dosage level of from about 1 mg to about 200 mg/day. Preferred daily doses generally will be from about 1 mg to about 50 mg/day. Preferred daily doses for the treatment OA are generally from about 5 mg to about 20 mg/day. A dosage of 10 mg/day may be preferred for some OA patients. Such a dosage may be given as a single dose or may be divided into two or three separate doses per day as necessary.

The present invention further provides a continuous dosing schedule whereby a unit dosage of arzoxifene is regularly administered according to the dosing interval of once-weekly (hereinafter “once-weekly dosing”) or twice-weekly (hereinafter “twice-weekly dosing”).

The term “once-weekly dosing” means that a unit dosage of arzoxifene is administered once during a week, i.e. one time during a seven day period, preferably on the same day of each week. In the once-weekly dosing regimen, the unit dosage is usually administered about every seven days. A nonlimiting example of a once-weekly dosing regimen would entail the administration of a unit dosage of arzoxifene every Sunday. It is preferred that the unit dosage is not administered on consecutive days, but the once-weekly dosing regimen can include a dosing regimen in which unit dosages are administered on two consecutive days falling within two different weekly periods. The term “once-weekly dosing” contemplated by the present invention, includes administration of a unit dosage with a periodicity ranging from about once every 3 (three) days to about once every 14 (fourteen) days.

The once-weekly dosing regimen can be useful for administering arzoxifene for the treatment of OA and/or for the treatment of other conditions that may benefit from the administration of arzoxifene. For example, the once-weekly dosing regimen can also be useful for the treatment of conditions described in the '474 patent. It may be preferred that the once-weekly dosing regimen is used for the treatment of osteoporosis or OA.

The term “twice-weekly dosing” means that a unit dosage of arzoxifene is administered twice during a week, i.e. twice during a seven day period, preferably on the same days of each week. In the twice-weekly dosing regimen, the unit dosage is usually administered about every four days. A nonlimiting example of a twice-weekly dosing regimen would entail the administration of a unit dosage of arzoxifene every Sunday and Thursday. It is preferred that the unit dosage is not administered on consecutive days, but the twice-weekly dosing regimen can include a dosing regimen in which unit dosages are administered on two consecutive days falling within two different weekly periods. The term “twice-weekly dosing” contemplated by the present invention, includes administration of a unit dosage with a periodicity ranging from about once every 2 (two) days to about once every 5 (five) days.

The twice-weekly dosing regimen can be useful for administering arzoxifene for the treatment of OA and/or for the treatment of other conditions that may benefit from the administration of arzoxifene. For example, the twice-weekly dosing regimen can also be useful for the treatment of conditions described in the '474 patent. It may be preferred that the twice-weekly dosing regimen is used for the treatment of osteoporosis or OA.

It may be preferred that the unit dose contains from 5 mg to 500 mg of arzoxifene. It may be especially preferred that the unit dosage form for twice-weekly dosing or once-weekly dosing contains from about 200 mg to about 350 mg. It may be preferred that a unit dose contains from about 275 mg to about 300 mg.

The methods and dosing regimens of the present invention are useful for inhibiting bone resorption and for treating and/or preventing abnormal bone resorption in a patient in need thereof. Although studies in laboratory animals using arzoxifene support that the half life of the molecule is consistent with daily dosing. Applicants were surprised to note that the half life of arzoxifene in human patients is longer than one day.

The arzoxifene less than daily administered unit dosage and dosing regimen is designed to administer a safely tolerated dosage that is greater than the previously expected optimal dosage, while providing an effective clinical benefit for a given interval of time. The human patient may initially experience a greater clinical exposure; however, the clinical benefit, with a pharmaceutically acceptable safety profile, generally continues as the arzoxifene is cleared from the patient's body. The less than daily dosing regimen may provide additional benefits to minimize the chance for undesired effects while preserving the clinical benefits from arzoxifene administration.

Patients administered arzoxifene using the less than daily dosing regimen are monitored for therapeutic benefit, physiological exposure to arzoxifene, and observed for drug safety profile signals.

In a further embodiment, the present invention relates to a kit for conveniently and effectively carrying out the dosing regimens of the present invention. Such kits are especially suited for the delivery of a solid oral form such as a tablet or capsule. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings with a calendar insert, designating the days in the treatment schedule in which the dosages are administered. Alternatively, placebo dosages, or dietary supplements, either in a form similar or distinct from the arzoxifene dosages, can be included to provide a kit in which a dosage is taken every day.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the present invention.

Biomarkers may be useful in assessing risk of developing a disease, establishing the diagnosis of the disease, predicting or monitoring the course of a disease, and/or assessing the response to a therapeutic intervention. For osteoarthritis (herein “OA”), biomarkers have heretofore shown poor ability to discriminate OA from absence of OA, largely because of significant variability in biomarker expression across normal and OA-affected individuals. Some biomarkers have been shown to be predictive of progression of radiographic joint space narrowing, which is considered a surrogate outcome for cartilage loss and disease progression. Correlations with progression of radiographically-assessed cartilage loss have been demonstrated for hyaluronic acid (HA), cartilage oligomeric matrix protein (COMP), the cross-linked C-terminal cross-linked telopeptides of type II collagen (CTXII), and the N-terminal peptides of type IIA procollagen (PIIANP). For some biomarkers or combinations of biomarkers, baseline values correlate with subsequent disease progression, whereas for others, the biomarker expression over time or change over time shows better correlation with radiographic outcomes. Inter-individual variability hampers the application of these biomarkers to the assessment and prognostication of particular subjects or patients, e.g., for the purpose of selecting subjects for trials of treatments intended to alter disease progression. The newer biomarker assays, e.g., CTXII and PIIANP, and combinations of assays, e.g., CTXII:PIIANP ratio, may minimize the variability and enhance the predictive power of biomarkers for OA progression. These biomarkers are attractive tools for early phase clinical studies because they appear to reflect changes in the articular cartilage, the putative “target tissue” of disease modifying treatments for OA. These changes appear to be part of the “final common pathway” of articular cartilage degeneration. Therefore, these biomarkers appear to have face, content, criterion, and construct validity with respect to articular cartilage degeneration. Furthermore, because these biomarkers can be assessed conveniently and repeatedly, i.e., with minimally invasive collection of urine and blood specimens rather than arthrocentesis or tissue biopsy, and are responsive to intervention over reasonable (˜3 months) periods of time, they appear to be suitable for exploration of OA structure-modifying effects. By comparison, assessment of articular cartilage degeneration by imaging technologies requires exposure to ionizing radiation (specially-positioned radiographs) and follow-up periods of ˜2 years or expensive and evolving magnetic resonance imaging (MRI) which may allow shortening of the follow-up interval to ˜6 months.

Guinea Pig Spontaneous Osteoarthritis Study

Hartley albino guinea pigs undergo spontaneous cartilage degeneration, and they have been used as a convenient small animal model system to study osteoarthritis (OA) and have potential physiologic relevance to the OA that spontaneously develops in humans. Disease-related morphologic changes observed in the guinea pigs are similar to the histopathologic alternations in the joints of humans with OA. Other significant similarities between the OA in Hartley guinea pigs and that in humans are also known. Huebner, Collagenase 1 and Collagenase 3 Expression in a Guinea Pig Model of Osteoarthritis, 41 Arthritis Rheum. 877-890 no. 5(May 1998). The Hartley guinea pig spontaneous model can be used to study the effects of Arzoxifene on OA.

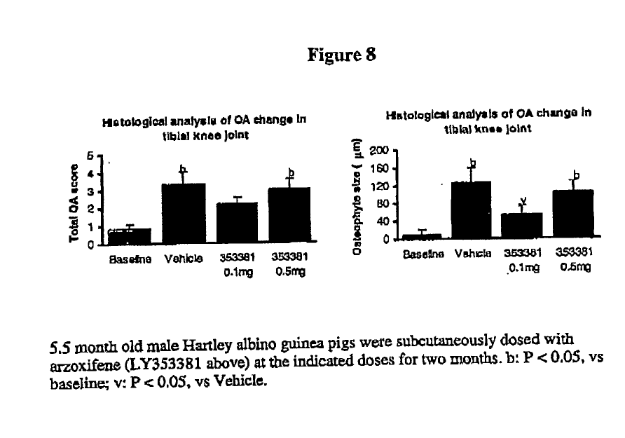

Hartley strain guinea pigs, approximately three month old males, are used for the study. (In one study, 5.5 month old males were used, as indicated by FIG. 8). The Hartley albino guinea pigs are provided ad lib access to food (Ca 1.1%, p 0.6%, Vitamin D 3400 IU/kg) and water. The guinea pigs are dosed subcutaneously with Arzoxifene at 0.03, 0.1, 0.3, and/or 0.5 mg/kg/d for about two to four months. The study generally includes 15 guinea pigs per treatment group, except for the baseline, which generally includes 10 guinea pigs. Urine and serum are to be collected at 0, 2, 4, 6, 12, and 16 weeks for biomarker evaluation. The right knee joints are harvested for histology and the left knee joint for surface osteoarthritis scoring.

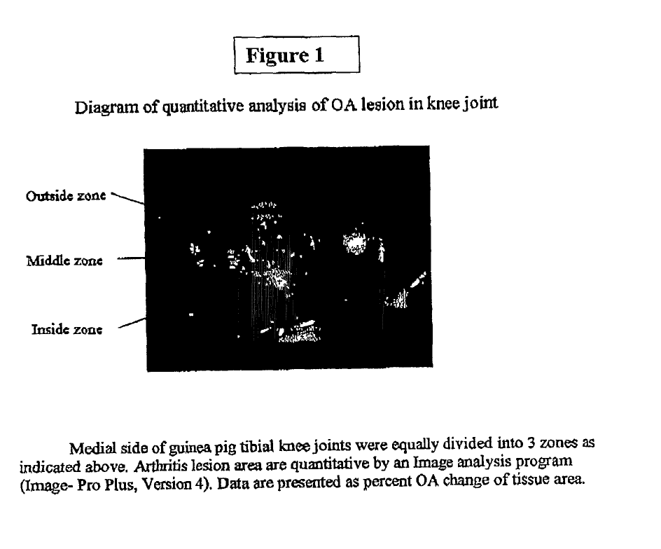

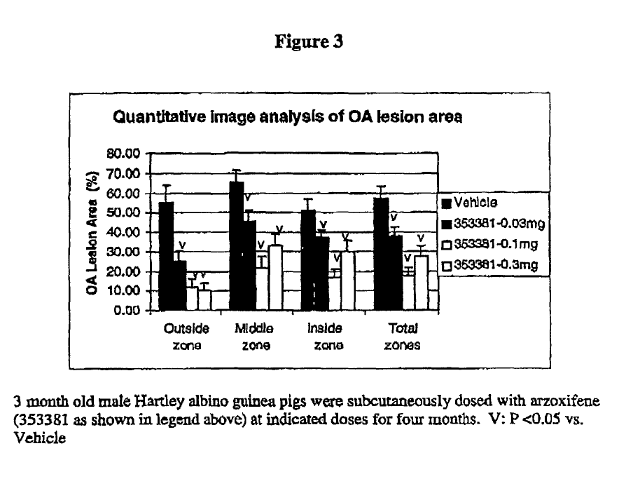

Data generated from the guinea pig model support that Arzoxifene has a positive effect on preventing or slowing the progression of OA. The effects observed on OA lesion areas by quantitative image analyses are illustrated by the graph in FIG. 3. The Zones of the lesion in the knee joint are illustrated by FIG. 1. Osteoarthritis changes are further evaluated by histologically quantitative analyses on paraffin embedded sections. The severities of joint damage are assessed for the depth of chondrocyte lesion and loss of proteoglycan on a score 0-5 for increasing severity on each third of the cartilage surface (Animla molde of osteoarthritis, A. M. Bendele (J Musculoskel Neuron Interact 2001: 1: 363-376)). Osteophyte development also evaluated by measuring the size.

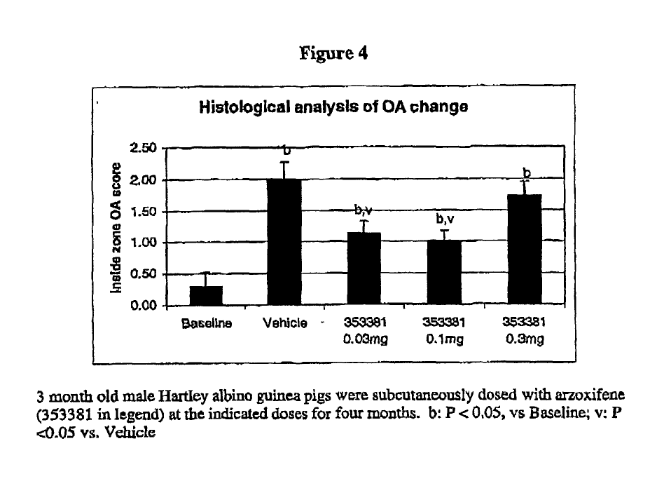

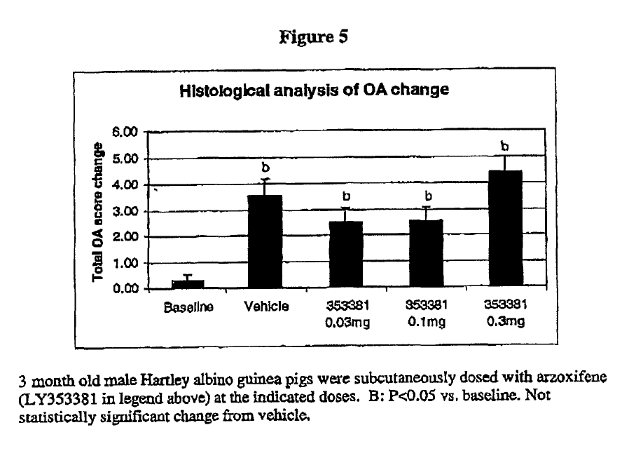

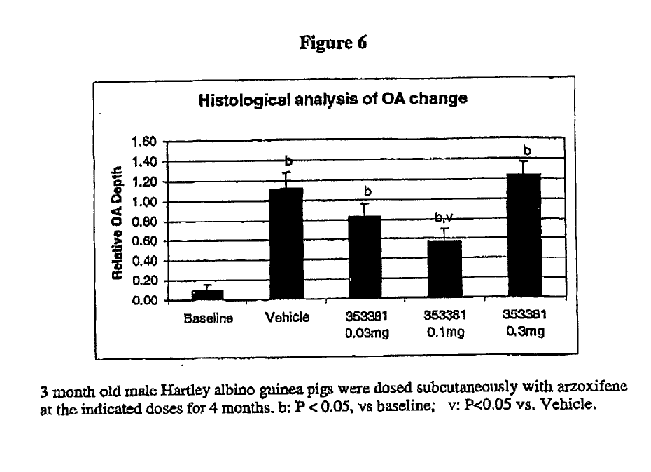

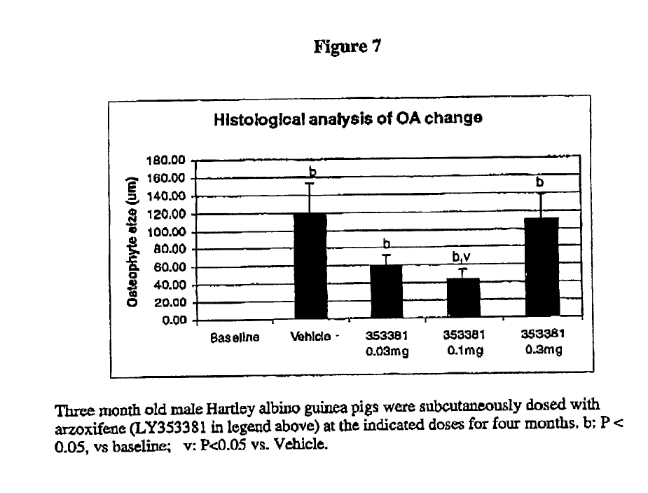

In one study, the 0.5 mg/kg/d dose of arzoxifene did not have a statistical effect on OA progression based on OA surface area and osteophyte development (as shown in FIG. 8). The lower doses of arzoxifene tested in the guinea pig study provided results (as shown by the FIGS. 4-7) to suggest that arzoxifene had a favorable effect on preventing or delaying the OA progress by reducing OA surface area and osteophyte development.

Intermittent Dosing Analysis of Arzoxifene in Ovariectomized Rats (Prevention Model)

Arzoxifene will be administered using several different dosing intervals for 5 weeks to 6 month-old rats, one week after ovariectomy.

Live phase: A total of about 78 female Sprague-Dawley rats (Harlan Sprague Dawley Inc) are used for this study design. 72 of the rats are ovariectomized (Ovx) at 6 months of age and 6 rats are used as sham-ovariectomized controls. Rats were maintained on a 12 hour light/dark cycle at 22° C. with ad lib access to food (TD 89222 with 0.5% Ca and 0.4% P, Teklad, Madison, Wis.) and water. Sham and Ovx controls are orally administered a vehicle of 20% hydroxy propyl-β-cyclodextrin (Aldrich Chemical Co). Arzoxifene (1 mg/kg) treated rats are dosed daily, every other day (QOD), every third day (Q3D), weekly on Monday (QW), or twice a week on Monday and Friday (M/F), as indicated. Each group contains 6 animals/grp and the treatment duration is about 5 weeks.

Analyses: Intact femora are removed and cleaned of soft-tissue and preserved in 50% ethanol/saline at 4° C. Femora are centered with respect to the QCT gantry (Research M, Stratec). A coronal scout scan of the specimen is generated first in 2 Dimension (2D) and then femora are scanned to measure bone mineral density (BMD, mg/cc), bone mineral content (BMC, mg), and cross-sectional area (mm2).

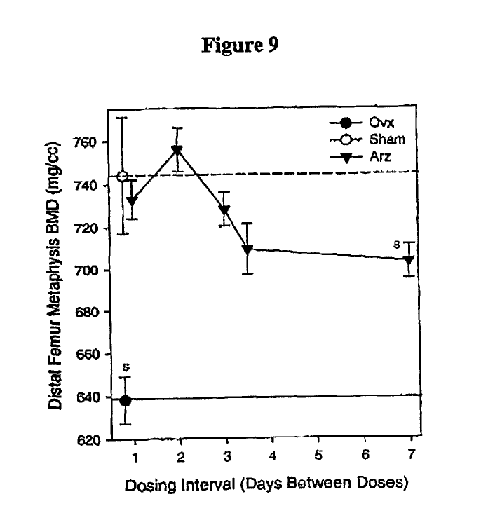

Results from one study using this experimental protocol are illustrated by FIG. 9, herein. Analysis of the distal femur metaphysis by QCT showed significant reduction of BMD for Ovx relative to Sham (P<0.05, Fishers PLSD). This loss in bone was prevented by daily, QOD, Q3D, and (M/F) treatment with arzoxifene. Weekly arzoxifene was only partially efficacious because QW BMD was intermediate between Ovx and Sham and less than Sham. Nevertheless, because the rate of bone turnover, metabolism, and pharmacokinetic profile (T1/2) are more than two times faster in the adult rat compared to adult humans, these studies suggest that arzoxifene will be efficacious to treat postmenopausal osteoporosis in adult humans, using a weekly, or less than daily (once weekly or more), oral dosing regimen.

Clinical Studies Overview

A Phase 2, multicenter, randomized, double-blind, placebo- and raloxifene-controlled study of arzoxifene enrolled 219 postmenopausal women with osteopenia (defined as bone mineral density [BMD] t-score at the lumbar spine and/or femoral neck >−2.5 and ≦−1). The study assessed the effects of arzoxifene compared with raloxifene HCl (60 mg/day) and placebo on biochemical markers of bone metabolism, cardiovascular disease risk markers, and on overall safety.

OA biomarkers were evaluated on a subset of samples from the osteopenia study, which, despite no selection of subjects for evidence of OA, demonstrated modest but significant biomarker differences between placebo and arzoxifene in COMP and human cartilage glycoprotein-39, also known as YKL-40. CTXII was not evaluated as the assay is validated for urine specimens, which were not available.

Clinical Study to Study OA

A 6-month double-blind placebo-controlled phase Ia “proof-of-concept” study is designed to primarily evaluate the effect of two doses of arzoxifene on biomarkers thought to correlate with OA progression, and secondarily to evaluate a “state of the art” MRI technique for demonstrating structural changes associated with OA. The subjects will be post-menopausal women with knee OA who, based upon clinical features, are anticipated to have a high probability of structural progression. Signs and symptoms data will be collected and will be explored for correlations with the treatment assignment, the biomarkers, and with the imaging studies.

Biomarkers

CTXII

CTXII was chosen as the primary outcome for this study. This biomarker indicates turnover of type II collagen, which is limited in its distribution within the body to the hyaline cartilage (diarthrodial joints, nasal/auricular/tracheal cartilage) and intervertebral discs. In a cross-section of patients with OA, CTXII correlated with the degree of cartilage loss in OA patients and differentiated OA patients from controls (the latter was shown for HA, COMP, and the C-terminal cross-linked telopeptides of type I collagen (CTXI), as well)[Garnero P et al, Ann Rheum Dis 2001; 60:619-626]. Baseline elevation of CTXII was “predictive” of rapid hip joint space loss [Garnero P et al Ann Rheum Dis 2003; 62:939-943] and correlated with hip and knee joint space narrowing (herein “JSN”) over prolonged follow-up (mean 6.6 years) in the observational Rotterdam Study [Reijman M et al, Arthritis Rheum 2003; 48:S683]. In the 3-year study of diacerhein in hip OA, CTXII and HA were independent predictors of JSN [Mazieres B et al, Arthritis Rheum 2003; 48:S683]. Interestingly, in this latter study, CTXII also correlated with pain and functional impairment [Garnero P et al, Arthritis Rheum 2003; 48:S291]. Of the patients participating in a 3-year study of glucosamine sulfate, a subset had baseline elevations greater than 1 standard deviation above the mean for normal controls (−30% of the study group). In this subset, declines in CTXII over the first 1 year correlated with reduction in JSN over 3 years [Christgau S et al, Clin Exp Rheumatol 2004; 22:36-42]. The trends for JSN-assessed treatment response were in the same direction for the subjects with lesser baseline CTXII values and for the whole study groups, but the differences were of lesser magnitude.

PIIANP

“Uncoupling” of type II collagen synthesis and degradation has been proposed based on the finding that the ratio of PIIANP (a presumed “synthesis” marker) to CTXII (a “turnover” or “degradation” marker) is more strongly predictive of progressive loss of knee cartilage over 1 year of follow-up, assessed by both radiography and arthroscopy, than is either marker alone [Garnero P et al, Arthritis Rheum 2002; 46:2613-2624]. Similarly, if both biomarkers were abnormal by >1 SD (PIIANP >1 S.D. below the mean, CTXII >1 S.D. above the mean), the predictive power was greater than for either biomarker alone. Importantly, elevated CTXII identified 13 of the 16 subjects (81%) with arthroscopically-assessed progression, albeit with a “true positive” rate of 13 out of 31 (42%); the combination of PIIANP and CTXII identified 10 progressors (63%), with a “true positive” rate of 67%. Subsequent work showed that immunoreactive PIIANP is present in the synovial fluid and serum as 80 and 100 kDa bands [Rousseau J-C et al, Osteoarthritis Cart 2004; 12:440-447], suggesting that it is not degraded significantly during its transit from the synovial to the serum space. The precise molecular structure of these moieties is not yet known, and the comparative concentrations in the serum and synovial fluid has not been reported.

COMP

COMP is found in higher concentrations in articular cartilage than in other tissues, but it is also synthesized in the synovium, and is currently considered by many to be indicative of synovitis [Vilim v et al, Osteoarthritis Cart 2001; 9:612-618]. Its serum concentrations at baseline are predictive of subsequent radiographically-assessed cartilage loss [Sharif M et al, Arthritis Rheum 1995; 38:760-767], and time-averaged COMP is more predictive of progression [Sharif M et al, Arthritis Rheum 2003; 48:S291, Vilim V et al, Osteoarthritis Cart 2002; 10:707-713]. Furthermore, COMP elevations appear to correlate with signs and symptoms of early hip more so than knee OA [Dragomir A D et al, Osteoarthritis Cart 2002; 10:687-691]. Serum COMP is affected by age, gender, BMI, weight gain [Loeser R F et al, Arthritis Rheum 2003; 48:S698] and activity level [Andersson M et al, Arthritis Rheum 2003; 48:S292]. Because of its wide variability between normals and OA, and between OA progressors and non-progressors, the discriminative power of COMP is less than for CTXII [Garnero P et al, Ann Rheum Dis 2001; 60:619-626].

HA

HA is synthesized and distributed in many tissues, although it is found in the highest concentrations in the cartilage and synovial fluid. It is degraded to shorter fragments in the presence of inflammation, exits the joint via the lymphatics, and is cleared from the systemic circulation by the liver. The correlation between synovial fluid and serum concentrations of HA is poor [Salisbury C, Sharif M, Ann Rheum Dis 1997; 56:558-561]. HA is variously considered to reflect synovitis [Manicourt D-H et al, J Rheumatol 1995; 22:262-269] or cartilage damage or repair [Otterness I G et al, Osteoarthritis Cart 2000; 8:180-185]. Because of its wide variability of serum HA between normals and OA, and between OA progressors and non-progressors, the discriminative power of HA is less than for CTXII [Garnero P et al, Ann Rheum Dis 2001; 60:619-626]. Nonetheless, baseline HA has been shown to predict radiographically-assessed [Sharma L et al, Arthritis Rheum 1998; 41:1233-1240, Sharif M et al, Ann Rheum Dis 2000; 59:71-74, Sharif M et al, Arthritis Rheum 1995; 38:760-767] and arthroscopically-assessed [Georges C et al, Arthritis Rheum 1997; 40:590-591] OA progression. However, in a “pivotal” 3-year study of glucosamine sulfate, decline of HA over the first one year of follow-up, but not baseline HA, correlated with 3-year radiographic OA progression [Bruyere O et al, J Rheumatol 2003; 30:1043-1050].

CTXI

Because the OA disease process is characterized by bony overgrowth (osteophytes) and sclerosis, particularly in the subchondral region, and because this bone remodeling is potentially involved in the disease progression, this clinical study examines a marker of type I collagen turnover, CTXI. CTXI levels have been shown to be variously increased [Bettica P et al, Arthritis Rheum 2002; 46:3178-3184] or decreased [Garnero P et al, Ann Rheum Dis 2001; 60:619-626] among OA patients.

Imaging

Radiography

While radiographic imaging of inter-bone distance (joint space) is currently the accepted surrogate measure of OA progression, it lacks accuracy, reproducibility, and sensitivity to change, and it represents only a restricted portion of the articular cartilage surface. It is generally accepted that JSN is best assessed during weight-bearing, with the knee in a semi-flexed position, and with the medial tibial plateau parallel to the x-ray beam. Multiple protocols have been proposed to achieve these aims. The Lyon Schuss and MTP techniques have been compared [Buckland-Wright J et al, J Rheumatol 1999; 26:2664-2674], as have fluoroscopically-positioned semi-flexed and full extension views [Buckland-Wright J C et al, Ann Rheum Dis 1995; 54:872-880], and fluoroscopically-positioned and Lyon Schuss views [Conrozier T G R et al, Arthritis Rheum 2003; 48:S487]. Unfortunately, fluoroscopically-positioned views, while theoretically advantageous, involve substantial additional radiation exposure and expense, and technician training and quality maintenance is difficult [Mazzuca S A et al, J Rheumatol 1999; 26:1359-1365]. An alternative fixed-flexion protocol has been compared to fluoroscopically-positioned views, with very similar reproducibility and accuracy [Peterfy C et al, Skeletal Radiol 2003; 32:128-132]. Computer-assisted image analysis [Duryea J et al, Osteoarthritis Cart 2003; 11:102-110] further minimized measurement errors. However, despite these enhancements, the duration of follow-up required to demonstrate changes in the rate of JSN is at least one year [Buckland-Wright J et al, Arthritis Rheum 2003; 48:S486], and the sample sizes required to demonstrate differences are large, assuming a “typical” annualized rate of JSN of ˜0.1 mm.

Magnetic Resonance Imaging (MRI)

MRI is substantially more sensitive to changes in the OA joint [Jones G et al, Osteoarthritis Cart 2004; 12:169-174, Dashti M et al, Scand J Rheumatol 2004; 33:87-93], but the specific measurement and measurement technique is not agreed upon. Most of the current MRI techniques utilize a 1.5 Tesla magnet, fat-suppressed imaging sequences, and assess the entire joint in 3 dimensions, allowing measurement of cartilage thickness and volume with measurement variability <3%. Dominant limbs have ˜5% greater cartilage volume and ˜4% greater cartilage thickness than the non-dominant limb of normal persons [Eckstein F et al, Osteoarthritis Cart 2002; 10:914-921]. Men have substantially greater cartilage volume than women, which is only partially mediated by body mass and bone size [Ding C et al, Rheumatology 2003; 42:1317-1323]. The sex differences become even greater above age 50 years. Healthy post-menopausal women have a decrease in tibial cartilage volume at a rate of 2.4%/year following cessation of hormone replacement therapy [Wluka A E et al, Ann Rheum Dis 2004; 63:444-449]. Correlations have been demonstrated between radiographic JSN and cartilage volume measurement [Cicuttini F M et al, Ann Rheum Dis 2001; 60:977-980], and between cartilage volume on the tibia and femur, suggesting that tibial cartilage volume, which is easier to measure than femoral or total knee cartilage volume, may suffice as a measure of OA progression [Cicuttini F M et al, Arthritis Rheum 2004; 50:94-97]. Loss of tibial cartilage volume has been shown to progress at a rate of ˜5%/year in knee OA patients [Wluka A E et al, Arthritis Rheum 2002; 46:2065-2072], with some evidence that progression is approximately linear during the early stages of OA. Because of small measurement error, 50 subjects per group should be adequate to demonstrate a between group cross-sectional difference in cartilage volume of 10% [Jones G et al Osteoarthritis Cart 2004; 12:169-174]. In a 6-month longitudinal study, 62 subjects per arm would be required to detect, with 80% power, a 50% reduction in the rate of progression, assuming the mean rate of progression is reasonably consistent at 4-5%/year and a measurement standard deviation of ˜4% [personal communication, Charles Peterfy M D, PhD, Synarc]. If a population of rapid progressors is evaluated (Ex: subjects with “bone marrow edema” (BME) or meniscal tears (vide infra), who are losing cartilage volume at a rate of 7+%/year with a standard deviation of ˜5%), then the study group sizes would decline to 54 subjects/group. However, if the detection limit is lowered to a 30% reduction in the rate of cartilage volume loss, the group sizes exceed 100.

Validation of MRI measurement against the actual anatomic measures on tissues excised at total knee arthroplasty has been performed [Graichen H et al, Arthritis Rheum 2004; 50:811-816]. Furthermore, even with use of a 0.2 Tesla MRI and a scoring system based upon arthroscopic grading of cartilage lesions, MRI was more sensitive to change than was arthroscopy and radiography [Pessis E et al, Osteoarthritis Cart 2003; 11:361-369]. As has been demonstrated with radiographic progression, rapid MRI progression in the knee is predicted by presence of a meniscal tear [Biswal S et al, Arthritis Rheum 2002; 46:2884-2892]. BME is an MRI feature that is not apparent on radiographs but is strongly associated with radiographically-assessed [Felson D T et al, Ann Intern Med 2003; 139:330-336] and MRI-assessed [Hunter D J et al, Arthritis Rheum 2003; 48:S428] progression of knee OA. The BME were strongly correlated with static limb alignment abnormalities, and with knee pain [Felson D T et al, Ann Intern Med 2001; 134:541-549]. An integrated scoring system that evaluates cartilage, osteophytes, subchondral bone, menisci, ligaments, bursae, synovium, and intraarticular loose bodies has been proposed recently [Peterfy C G et al, Osteoarthritis Cart 2004; 12:177-190]. The intra-class correlations between readers was >0.90 for cartilage and osteophytes, with lesser agreement on BME and synovium scoring. The scoring system for cartilage is derived from arthroscopic scoring systems, taking into account the percentage of cartilage surface that is involved and the depth of involvement, but also includes intra-cartilaginous signal abnormalities. This allows assessment of “lesion severity” rather than cartilage volume or thickness only. A technological enhancement of future interest is the ability to detect tibiofemoral intracartilaginous T2 signal abnormalities using a 3 Tesla MRI [personal communication, Charles Peterfy M D, PhD, Synarc].

MRI is not performed with knee loading, which does have a modest effect on cartilage volume, and perhaps more importantly, on cartilage contour. To minimize variability, the MRI images should be acquired at approximately the same time of day and day of the week and with maintenance of similar levels of physical activity (to minimize differences in intra-cartilaginous fluid re-distribution associated with load-bearing, e.g., with standing and walking). BME might also change with level of physical activity. Joint space width can be affected, although the sensitivity of the radiographic technique usually does not allow demonstration of the effect.

Preliminary evidence suggests that symptoms may correlate with knee OA MRI cartilage findings [Raynauld J-P et al, Arthritis Rheum 2004; 50:476-487, Wluka A E et al, Ann Rheum Dis 2004; 63:264-268]. Further study will be required to show whether these correlations are stronger and more consistent than has been demonstrated with knee radiography [Bruyere O et al, Scand J Rheumatol 2002; 31:13-16, Lethbridge-Cejku m et al, Arthritis Care Res 1995; 8:182-188, McAlindon T et al, Ann Rheum Dis 1993; 52:258-262, Ciccutini F et al, Osteoarthritis Cart 1996; 4:143-147].

The biomarkers have substantial variability in their “normal” ranges and intra-subject inter-assay variability, lack specificity for OA and for specific joint sites of involvement, and are affected by the conditions of sample collection and by demographics. New biomarker assays may improve on some of these deficiencies, but particularly in the refinement of the specificity of the markers for biochemical/pathophysiological processes.

Radiographic imaging is the accepted surrogate endpoint for structural progression of OA. It is clear that meticulous adherance to performance techniques that include a semi-flexed knee position, adjusted leg rotation, beam angle, and magnification correction are mandatory for radiographic measurement precision. MRI is advancing, and has great potential for measuring cartilage volume with much better precision and reproducibility. Furthermore, depending on the specific measurement technique utilized, MRI can simultaneously assess changes in other joint tissues, including the bone, capsule, ligaments, and synovial fluid, which may, individually or as an aggregate, indicate the impact of an intervention.

Clinical Study

A study to demonstrate the clinical effects of arzoxifene on the treatment and/or progression of OA is designed as follows. The study is a 6-month, multicenter, Phase IIa, randomized, double-blind, parallel, placebo-controlled trial in postmenopausal women with radiographically-documented knee OA. The study population will be ≧2 years postmenopausal. Approximately 75 postmenopausal women will be enrolled in this study at an approximately 1:1:1 ratio for azoxifene 5 mg:arzoxifene 20mg:placebo.

Screening safety laboratory studies and standing semi-flexed x-rays of both knees will be performed. Only subjects who meet all eligibility criteria after completion of these tests will be asked to return for randomization to study treatment. At Visit 2, eligible subjects, stratified according to their standing knee alignment (0-5° varus versus 5-10° varus) of their radiographically more severely affected knee (index knee) will be enrolled and randomized to receive either arzoxifene 5 mg/day, arzoxifene 20 mg/day, or placebo during the double-blind treatment phase. Subjects will continue taking the blinded drug treatment assignment throughout the remainder of the study.

Treatment Phase

Subjects will receive double-blind study medication for 26 weeks throughout the treatment phase. Biomarkers will be assessed at baseline and after 26±2 weeks of study treatment. MRI of the index knee will be assessed at baseline and at 26±2 weeks of study treatment. Biomarker specimens will also be collected after 3±1 and 6±1 weeks of study treatment. OA signs and symptoms will be assessed at each study visit.

Study Population

Study subjects are women at least 2 years postmenopausal AND between 50 and 70 years old, inclusive. At study entry, women must have knee pain and radiographic evidence of knee OA. Subjects with clinical features of hip OA must not have radiographic evidence of severe hip joint space loss.

In clinical use, the specific doses of arzoxifene will, of course, be determined by the particular circumstances surrounding the case. Similarly, the route of administration is a factor determined by the specifics of each case. Thus, the exact dose and route of administration are best determined by the attending physician.

1. A method for treating osteoarthritis in a mammal, comprising administering to a mammal in need thereof, an effective amount of a compound of Formula I: 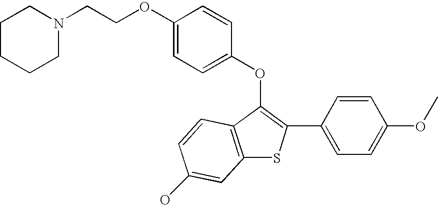
or a pharmaceutically acceptable salt or solvate thereof.

2. A method as claimed by claim 1 wherein the mammal is a human patient.

3. A method as claimed by claim 2 wherein the human patient is a woman.

4. A method as claimed by claim 2 wherein the human patient is a man.

5. A method as claimed by claim 1 wherein the human patient has been clinically diagnosed as being in need of treatment for osteoarthritis.

6. A method as claimed by claim 1 wherein the mammal is selected from the group consisting of horses and companion animals.

7. A method as claimed by claim 1 wherein the compound of Formula I is arzoxifene.

8. A method as claimed by claim 1 wherein the compound is a pharmaceutically acceptable salt.

9. A method as claimed by claim 8 wherein the salt is the hydrochloride salt.

10. A method as claimed by claim 1 wherein the compound is a solvate.

11. A method as claimed claim 1 wherein the treatment alleviates the signs and symptoms of osteoarthritis.

12. A method as claimed by claim 1 wherein the treatment slows the progression of osteoarthritis.

13. A method as claimed by claim 1 wherein the treatment reverses the progression of osteoarthritis.

14. A method as claimed by claim 1 wherein the effective amount is administered as a unit dosage form.

15. A method as claimed by claim 1 wherein the effective amount is from about 1 mg to about 50 mg per day.

16. A method as claimed by claim 1 wherein the effective amount is from about 250 mg to about 300 mg.

17. A method as claimed by claim 1 wherein the effective amount is administered using a dosing regimen selected from the group consisting of daily dosing and less than daily dosing.

18. A method as claimed by claim 17 wherein the dosing regimen is daily dosing.

19. A method as claimed by claim 18 wherein the dosing regimen is less than daily dosing.

20. A method as claimed by claim 19 wherein the dosing regimen is once-weekly dosing.

21. A method as claimed by claim 19 wherein the dosing regimen is twice-weekly dosing.

22. (canceled)

23. A method for treating osteoporosis in a mammal in need thereof comprising administering to said mammal, a pharmaceutically effective amount of arzoxifene, or a pharmaceutically acceptable salt or solvate thereof, as a unit dosage according to a continuous schedule having a dosing interval selected from the group consisting of once-weekly dosing and twice-weekly dosing.

24. A method as claimed by claim 23 wherein the arzoxifene salt is the hydrochloride.

25. A method as claimed by claim 23 wherein the mammal is a human.

26. A method as claimed by claim 23 wherein the unit dosage comprises from about 20 mg to about 300 mg arzoxifene.

27. A method as claimed by claim 23 wherein the unit dosage comprises from about 90 mg to about 300 mg arzoxifene.

28. A method as claimed by claim 23 wherein the unit dosage comprises about 275 mg arzoxifene.

29. A method as claimed by claim 23 wherein the dosing regimen is once-weekly dosing.

30. A method as claimed by claim 23 wherein the dosing regimen is twice-weekly dosing.

31. A method for breast cancer risk reduction in a mammal in need thereof comprising administering to said mammal, a pharmaceutically effective amount of arzoxifene, or a pharmaceutically acceptable salt or solvate thereof, as a unit dosage according to a continuous schedule having a dosing interval selected from the group consisting of once-weekly dosing and twice-weekly dosing.

32. A method as claimed by claim 31 wherein the arzoxifene salt is the hydrochloride.

33. A method as claimed by claim 31 wherein the mammal is a human.

34. A method as claimed by claim 32 wherein the unit dosage comprises from about 20 mg to about 300 mg arzoxifene.

35. A method as claimed by claim 32 wherein the unit dosage comprises from about 90 mg to about 300 mg arzoxifene.

36. A method as claimed by any claim 32 wherein the unit dosage comprises about 275 mg arzoxifene.

37. A method as claimed by claim 31 wherein the dosing regimen is once-weekly dosing.

38. A method as claimed by claim 31 wherein the dosing regimen is twice-weekly dosing.

39. A method for preserving bone mineral density in a mammal in need thereof comprising administering to said mammal, a pharmaceutically effective amount of arzoxifene, or a pharmaceutically acceptable salt or solvate thereof, as a unit dosage according to a continuous schedule having a dosing interval selected from the group consisting of once-weekly dosing and twice-weekly dosing.

40. A method as claimed by claim 39 wherein the method for preserving bone mineral density comprises inhibiting bone resorption.

41. A method as claimed by claim 40 wherein the arzoxifene salt is the hydrochloride.

42. A method as claimed by claim 40 wherein the mammal is a human.

43. A method as claimed by claim 40 wherein the unit dosage comprises from about 20 mg to about 300 mg arzoxifene.

44. A method as claimed by claim 40 wherein the unit dosage comprises from about 90 mg to about 300 mg arzoxifene.

45. A method as claimed by claim 40 wherein the unit dosage comprises about 275 mg arzoxifene.

46. A method as claimed by claim 40 wherein the dosing regimen is once-weekly dosing.

47. A method as claimed by claim 40 wherein the dosing regimen is twice-weekly dosing.

48. A kit comprising at least one pharmaceutically effective unit dosage of arzoxifene, or a salt or solvate thereof, and label instructions to administer said unit dosage according to a continuous schedule having a dosing interval selected from the group consisting of once-weekly dosing and twice-weekly dosing.

49. A kit as claimed by claim 48 wherein the unit dosage comprises about 20 mg to about 300 mg arzoxifene.

50. A kit as claimed by claim 48 wherein the unit dosage comprises about 90 mg to about 300 mg arzoxifene.

51. A kit as claimed by claim 48 wherein the unit dosage comprises about 275 mg arzoxifene.

52. A kit as claimed by claim 48 wherein the label instructions further state that arzoxifene is administered to a mammal.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)
